(19) United States
(12) Patent Application Publication
(10) Pub. No.: US 2025/0249193 A1
(43) Pub. Date: Aug. 7, 2025

(54) AIRWAY DEVICE

(71) Applicant: KB Pro, Inc., Phoenix, AZ (US)

(72) Inventors: Cody Birch, Phoenix, AZ (US); Thomas G. Kotoske, Phoenix, AZ (US); Paul A. Fehrenbacher, Deer Park, IL (US); Joseph H. Voss, Star, ID (US); Scott F. Imus, Phoenix, AZ (US); Eric Wardrip, Rancho Santa Fe, CA (US); Adam Stephen Troness, Dellwood, MN (US); Daniel E. Roush, Niles, IL (US); Gregory Lyons, Carmel, IN (US)

(73) Assignee: KB Pro, Inc., Phoenix, AZ (US)

(21) Appl. No.: 19/186,507

(22) Filed: Apr. 22, 2025

Related U.S. Application Data
(63) Continuation-in-part of application No. 18/349,884, filed on Jul. 10, 2023.
Application No. 18/349,884 is a continuation of application No. 16/803,880, filed on Feb. 27, 2020, now Pat. No. 11,744,970.
(60) Provisional application No. 63/637,239, filed on Apr. 22, 2024.
(60) Provisional application No. 63/684,576, filed on Aug. 19, 2024.
(60) Provisional application No. 62/892,452, filed on Aug. 27, 2019.

(51) Int. Cl. A61M 16/04; A61M 16/08

(52) U.S. Cl. CPC A61M 16/0402; A61M 16/0434; A61M 16/0461; A61M 16/0486; A61M 16/085; A61M 2230/43

(57) ABSTRACT

An airway measurement device including a flexible tube having an opening at one end, a monitored fluid unit connected to the flexible tube on an end opposite the opening, an oxygen supply connection unit adjacent to the monitored fluid unit with the oxygen supply connection unit having a venting unit positioned parallel to the flexible tube, and a gripping unit on the bottom the monitored fluid unit and the oxygen supply connection unit.

### CROSS-REFERENCE TO RELATED APPLICATION

This application claims the benefit of U.S. Provisional Patent Application Ser. No. 63/637,239, filed on Apr. 22, 2024, and U.S. Provisional Patent Application Ser. No. 63/684,576, filed on Aug. 19, 2024, and is a continuation in part of U.S. patent application Ser. No. 18/349,884 filed Jul. 10, 2023, which is a continuation of U.S. patent application Ser. No. 16/803,880 filed Feb. 27, 2020, which claims the filing date benefit of U.S. Provisional Patent Application Ser. No. 62/892,452, filed on Aug. 27, 2019, and titled “AIRWAY ADJUNCT DEVICE,” the entire contents of which are hereby incorporated by reference.

### BACKGROUND

### Technical Field

The present disclosure relates generally to human airway devices. In particular, this disclosure relates to medical devices for opening and/or maintaining a patient's airway.

### Description of Related Art

Airway management devices are commonly used in the medical field for anesthesia and sedation cases where the healthcare provider does not use intubation or deep sedation, but where the conditions call for the patient to be semi-unconscious or unconscious during the procedure. Such airway devices include oropharyngeal airways and nasopharyngeal airways. In use, an airway device is inserted into the patient's mouth or nose, where it may help to maintain the airway free from obstruction due to relaxed airway musculature and the tongue.

In practice, airway devices have been increasingly used for procedures in office-based practice, in emergency rooms, for MRI patients, and for other cases where moderate to deep sedation with intravenous anesthetics are administered. Currently, a common practice is for healthcare providers to tape or secure tubing on, in, or near the airway device in order to analyze carbon dioxide exhaled by the patient and/or administer oxygen to the patient. However, taping or otherwise securing tubes on the airway device or the patient can be inefficient and may result in insecure or obstructive lines on or around the patient.

### SUMMARY

In one embodiment, an airway adjunct is disclosed. The airway adjunct includes a gas administration tube and a gas sampling tube. The gas administration tube has a first internal terminal end and a first port. The first port is at the opposite end of the gas administration tube from the first internal terminal end. The gas sampling tube has a second internal terminal end and a second port. The second port is at an opposite end of the gas sampling tube from the second internal terminal end. The second internal terminal end is offset at least one millimeter from the first internal terminal end in a longitudinal direction away from the second port. The longitudinal direction is defined as a direction parallel to an airway flow direction of the airway adjunct.

In another embodiment, a method of maintaining a patient's airway is disclosed.

The method includes providing an airway assembly and inserting the airway assembly into the patient's airway. The airway assembly includes a gas administration tube and a gas sampling tube. The gas administration tube has a first internal terminal end and a first port. The first port is at the opposite end of the gas administration tube from the first internal terminal end. The gas sampling tube has a second internal terminal end and a second port. The second port is at an opposite end of the gas sampling tube from the second internal terminal end. The second internal terminal end is offset at least one millimeter from the first internal terminal end in a longitudinal direction away from the second port. The longitudinal direction is defined as a direction parallel to an airway flow direction of the airway assembly.

In another embodiment, an airway assembly is disclosed. The airway housing includes a housing, a gas administration tube, and a gas sampling tube. The gas administration tube has a first internal terminal end and a first port. The first port is at the opposite end of the gas administration tubefrom the first internal terminal end. The gas sampling tube has a second internal terminal end and a second port. The second port is at an opposite end of the gas sampling tube from the second internal terminal end. The second internal terminal end is offset at least one millimeter from the first internal terminal end in a longitudinal direction away from the second port. The longitudinal direction is defined as a direction parallel to an airway flow direction of the airway assembly.

The present disclosure will now be described more fully with reference to the accompanying drawings, which are intended to be read in conjunction with both this summary, the detailed description, and any preferred or particular embodiments specifically discussed or otherwise disclosed. This disclosure may, however, be embodied in many different forms and should not be construed as limited to the embodiments set forth herein; rather, these embodiments are provided by way of illustration only so that this disclosure will be thorough, and fully convey the full scope of the invention to those skilled in the art.

### BRIEF DESCRIPTION OF THE DRAWINGS

Non-limiting and non-exhaustive embodiments of the present disclosure are described with reference to the following figures, wherein like reference numerals refer to like parts throughout the various views unless otherwise specified.

FIG. 1 is a top, perspective view of an airway adjunct according to one embodiment of the present disclosure.

FIG. 2 is a bottom, perspective view of an airway adjunct according to embodiments of the present disclosure;

FIG. 3 depicts an airway assembly according to one embodiment of the present disclosure;

FIG. 4 is a side, perspective view of an airway adjunct according to another embodiment of the present disclosure;

FIG. 5 is a bottom, perspective view of an airway adjunct according to another embodiment of the present disclosure;

FIG. 6 is a reverse, side perspective view of an airway adjunct according to another embodiment of the present disclosure;

FIG. 7 depicts an airway assembly according to another embodiment of the present disclosure;

FIG. 8 is a front, perspective view of an airway adjunct comprising tubes joined by a web according to one embodiment of the present disclosure;

FIG. 9 is a rear perspective view of an airway adjunct comprising tubes joined by a web according to one embodiment of the present disclosure;

FIG. 10 depicts an airway assembly comprising an airway adjunct inserted into an airway device;

FIG. 11 depicts an integral airway device comprising ports according to one embodiment of the present disclosure;

FIG. 12 is a depiction of a carbon dioxide waveform where an airway device has a gas sampling port that is approximately even with an oxygen administration port; and

FIG. 13 is a depiction of a carbon dioxide waveform where an airway device has a gas sampling port that is longitudinally offset from an oxygen administration port according to various embodiments of the present disclosure;

FIG. 14 depicts a side view of an adjunctive airway monitoring device;

FIG. 15 depicts a perspective view of the AAMD device;

FIG. 16 depicts a perspective view of the AAMD device;

FIG. 17 depicts a perspective view of the AAMD device 1400 connected to an OPA device;

FIG. 18 depicts a perspective view of an OPA device;

FIG. 19 depicts a cutaway view of the OPA device;

FIG. 20 depicts another embodiment of an AAMD;

FIG. 21 depicts a side view of the AAMD′

FIG. 22 depicts a top view of the AAMD;

FIG. 23A depicts a perspective view of an endoscopy bite block;

FIG. 23B depicts a top view of bite block;

FIG. 24 depicts an airway device;

FIG. 25 depicts a side perspective view of an airway device;

FIG. 26 depicts a perspective view of a simple bite block;

FIG. 27 depicts a process of gathering and storing information on a surgical procedure; and

FIG. 28 depicts aa process performed to model a surgical procedure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. A Iso, common but well-understood elements that are useful or necessary in a commercially feasible embodiment are often not depicted in order to facilitate a less obstructed view of these various embodiments of the present disclosure.

### DETAILED DESCRIPTION

In the following description, reference is made to exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the spirit and scope of the present disclosure. The following detailed description is, therefore, not to betaken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, or characteristics may be combined in any suitable combinations and/or sub-combinations in one or more embodiments or examples.

For various types of procedures, current airway devices may lack the ability to provide carbon dioxide analysis and administration of oxygen to a patient. Embodiments of the present disclosure comprise ports for carbon dioxide analysis, oxygen administration, and/or additional analysis, air administration, or other access functionalities. In various embodiments, the ports comprise sockets, apertures, connectors, or other types of openings for fluid communication.

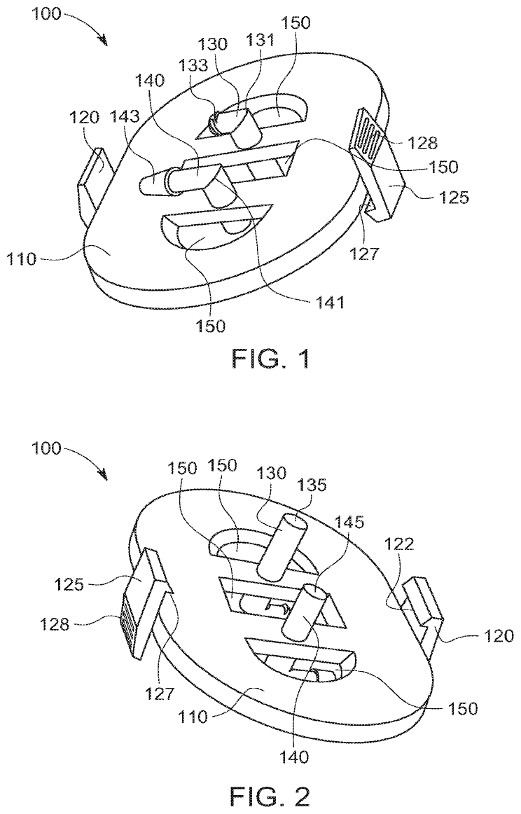

Referring to FIGS. 1-2, one embodiment of the present disclosure comprises an airway adjunct 100. In embodiments, airway adjunct 100 may be secured to an airway device (not shown in FIG. 1) to provide an exhalation gas sampling tube, a gas administration tube, and/or additional access as may be useful. In one embodiment, airway adjunct 100 comprises an essentially flat, disc-like body 110. In other embodiments, airway adjuncts have a body comprising a variety of shapes or geometries. In some embodiments, airway adjunct body 110 has a shape and size to approximately overlap the opening of an airway device to which the airway adjunct 100 may be attached. Some embodiments of airway adjunct 100 may include openings 150 in the body 110 to allow for ventilation through the airway adjunct 100 as the patient breathes or for insertion of suction tubing (not shown).

Embodiments of airway adjunct 100 comprise clamping tabs 120, 125 that are adapted to secure airway adjunct 100 to an airway device. In one embodiment, clamping tabs 120, 125 can provide a clamping force onto an airway device to which airway adjunct 100 is secured. In one embodiment, clamping tabs 120, 125 comprise a corresponding inner-facing step 122, 127 (shown in FIG. 2) to secure to an annular rim on an airway device and maintain airway adjunct 100 positioned on said airway device. In embodiments, clamping tabs 120, 125 are adapted to flex, with the stepped end flexing outward as airway adjunct 100 is affixed to and/or removed from the airway device. Embodiments of clamping tabs 120, 125 comprise knurled surface 128 to provide grip as a user squeeze clamping tabs 120, 125 in order to move stepped ends outward to release steps 122, 127 from the annular rim on an airway device and thereby remove airway adjunct 100 from said airway device.

Embodiments of airway adjunct 100 comprise a gas sampling tube 130 and gas administration tube 140. In various embodiments, gas sampling tube 130 comprises port 133. In embodiments, port 133 is located at a distal end of gas sampling tube 130. Embodiments of port 133 comprise a connector compatible for carbon dioxide monitoring, such as a T-Connector or Luer-style connector, or other types of connectors and/or tubing that may be used with capnography equipment. In other embodiments, port 133 is adapted to provide access for other types of expiration monitoring equipment or devices.

In various embodiments, port 133 is adapted to connect to a tubing, hose, and the like to provide fluid communication from gas sampling tube 130 to a carbon dioxide monitoring machine (not shown). According to various embodiments, the carbon dioxide monitoring machine monitors exhaled carbon dioxide via the gas sampling tube 130. For example, the carbon dioxide monitoring machine may comprise an end-tidal carbon dioxide monitoring machine. In various embodiments, port 133 connects directly or indirectly to an apparatus adapted to measure a partial pressure of carbon dioxide and/or maximal concentration of carbon dioxide at the end of an exhaled breath. According to various embodiments, monitoring equipment connected to port 133 may be utilized to measure vital signs of the patient.

In some embodiments, gas administration tube 140 comprises port 143. In embodiments, port 143 is located at a distal end of gas administration tube 140. Embodiments of port 143 comprise a connector compatible for administration of oxygen or other gas to be administered to a patient. Embodiments of port 143 are adapted to connect to tubing that may connect to an oxygen source.

In embodiments of the present disclosure, tubes 130, 140 each include an elbow bend 131, 141 of approximately ninety degrees. According to other embodiments, elbow bends have angles other than ninety degrees. Some embodiments of airway adjunct 100 comprise tubes 130, 140 having no bend. In embodiments, elbow bends 131, 141 are situated such that when airway adjunct 100 is attached to and/or inserted within an airway device in a patient's airway, ports 133, 143 are directed toward the patient's chin or the patient's nose. In other embodiments, elbow bends 131, 141 are angled laterally, to the side of the patient's face. In other embodiments, elbow bends 131, 141 are angled at a combination of lateral and medial directions. In some embodiments, elbow bends 131, 141 do not comprise mutually corresponding angles.

Referring to FIG. 2, embodiments of airway adjunct 100 comprise internal terminal ends 135, 145 of tubes 130, 140, respectively. According to embodiments, exhaled carbon dioxide and/or other exhaled gases may pass into internal terminal end 135, through gas sampling tube 130, through a tube connected thereto via port 133, and into a gas sampling machine as described above. In some embodiments, oxygen or other gas administered to a patient may flow from a gas source such as one or more tanks, through a supply tube, into gas administration tube 140 via port 133, out internal terminal end 145, and into the patient's airway.

Embodiments of the present disclosure include internal terminal ends 135, 145, each defining a different end position of tubes 130, 140. Throughout the present disclosure, the respective lengths of tubes 130, 140 leading to internal terminal ends 135, 145 may be defined in a relative sense by comparing the distance between each internal terminal end 135, 145, regardless of the actual length of tubes 130, 140. Such a measurement of the distance between the internal terminal ends 135, 145 may be made along a line parallel to one or both tubes 130, 140 near their respective internal terminal ends 135, 145. In other words, a statement that internal terminal end 135 is approximately one millimeter longer than internal terminal end 145 can be interpreted to mean that internal terminal end 135 extends approximately one millimeter farther into the patient's airway and/or into an airway device to which airway adjunct 100 is attached. Alternatively, the respective length of each internal terminal end 135, 145 may be defined as a distance that the respective portions of tubes 130, 140 extend from body 110.

In embodiments, a difference in position between internal terminal ends 135, 145 may allow the patient's exhaled breath to be sampled more accurately due to reduced diffusion with the oxygen and/or other gas administered from gas administration tube 140 via internal terminal end 145. In some embodiments, internal terminal end 135 extends farther into the patient's airway than internal terminal end 145, so that administered oxygen or other gas exiting from internal terminal end 145 might not mix with the patient's exhaled breath or may only minimally mix therewith.

According to some embodiments, the positional differential between internal terminal ends 135 and 145 is defined as a ratio between the respective tubes 130, 140. For example, in one embodiment, the portion of tube 130 between body 110 and internal terminal end 135 is fifty percent longer than the corresponding length of tube 140 between body 110 and internal terminal end 145. In the present disclosure, the respective portions of tubes 130, 140 between body 110 and respective internal terminal ends 135, 145 may be referred to as the “interior portion.” In other examples, the interior portion of tube 130 is forty to sixty percent longer than the interior portion of tube 140. In other examples, the interior portion of tube 130 is thirty to forty percent longer than the interior portion of tube 140. In other examples, the interior portion of tube 130 is at least ten percent longer than the interior portion of tube 140. In other examples, the interior portion of tube 130 is at least five percent longer than the interior portion of tube 140.

According to some embodiments, the positional differential between internal terminal ends 135 and 145 is defined as an absolute offset difference therebetween. For example, in one embodiment, internal terminal end 135 is offset approximately one millimeter from internal terminal end 145. In other examples, internal terminal end 135 is offset approximately one to two millimeters from internal terminal end 145. In other examples, internal terminal end 135 is offset approximately two to four millimeters from internal terminal end 145. In other examples, internal terminal end 135 is offset at least approximately one millimeter from internal terminal end 145. In the present disclosure, the term “approximately” may refer to a range within ten percent of the stated value.

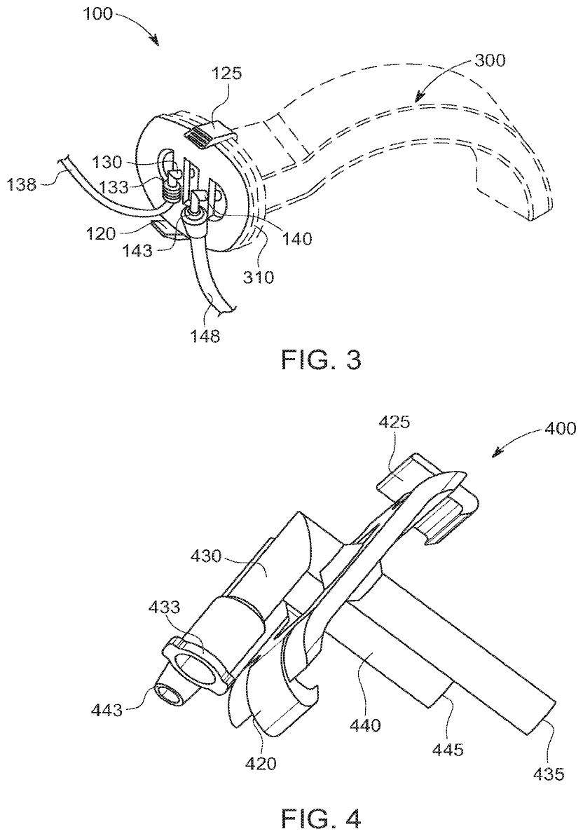

Various embodiments of the present disclosure are adapted to removably attach to an airway device to form an airway assembly. FIG. 3 depicts airway adjunct 100 attached to an oropharyngeal airway device 300. Various embodiments are adapted to attach to and/or on a variety of types of airway devices. In various embodiments, tube 138 may provide communication from gas sampling tube 130 to a gas sampling device. In various embodiments, tube 148 may provide communication from an administered gas source, such as an oxygen tank or supply line, to gas administration tube port 140.

While airway adjunct 100 is attached to an oropharyngeal airway device 300 as shown in FIG. 3, interior portions of tubes 130, 140 extend into the body or housing of the airway device. Clamping tabs 120, 125 secure airway adjunct 100 to collar 310 of oropharyngeal airway device 300.

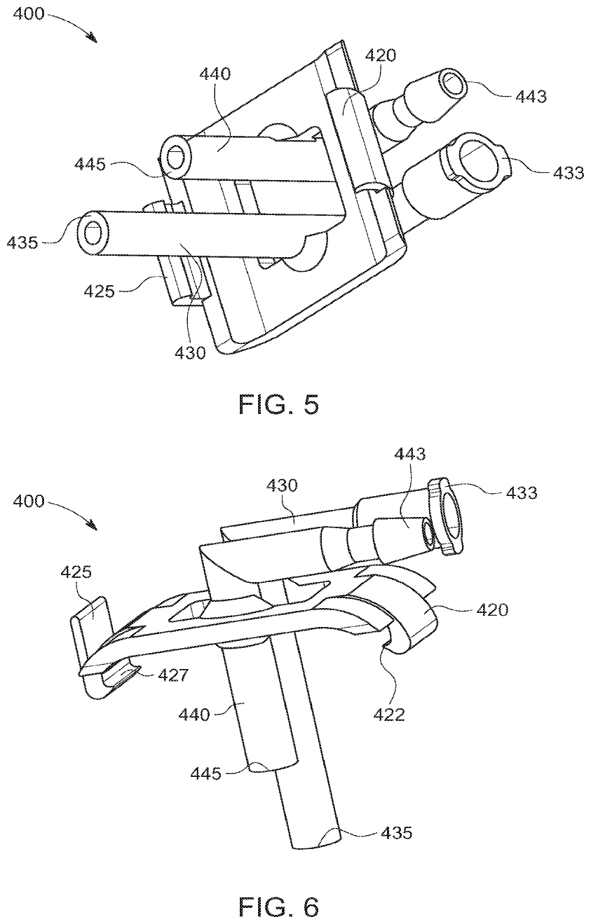

Referring to FIGS. 4-6, one embodiment of an airway adjunct 400 is depicted. As set forth above with respect to airway adjunct 100, airway adjunct 400 comprises gas sampling tube 430 and gas administration tube 440. In embodiments, gas sampling tube 430 comprises port 433 and internal-facing end 435. In embodiments, gas administration tube 440 comprises port 443 and internal facing end 445.

One embodiment of airway adjunct 400 comprises clamping tabs 420, 425. In embodiments, clamping tabs 420, 425 comprise a corresponding inner-facing step 422, 427 to secure to an annular rim on an airway device and maintain airway adjunct 400 positioned on said airway device. In embodiments, clamping tab 425 is adapted to flex, with the stepped end flexing outward as airway adjunct 400 is affixed to and/or removed from the airway device. Embodiments of clamping tab 425 comprise a knurled surface (not shown) to provide grip as a user squeeze clamping tab 425 in order to move inner-facing step 427 outward to release steps 422, 427 from the annular rim on an airway device and thereby remove airway adjunct 400 from said airway device.

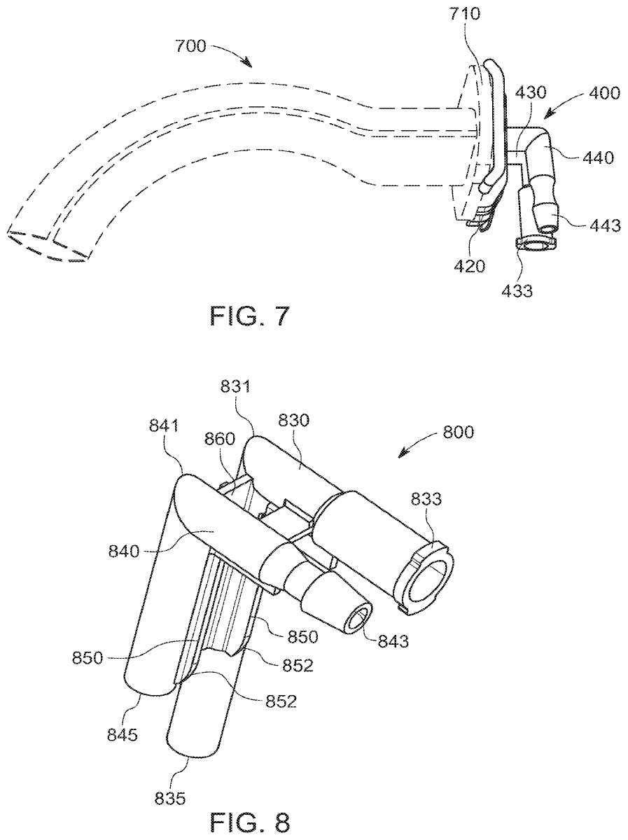

FIG. 7 depicts airway adjunct 400 attached to an oropharyngeal airway device 700, thereby forming an airway assembly. As shown, airway adjunct 400 is held to oropharyngeal airway device 700 by clamping tabs 420, 425 secured to rim 710 or collar of oropharyngeal airway device 700. In the configuration depicted in FIG. 7, internal-facing ends 435, 445 terminate within the body of oropharyngeal airway device 700.

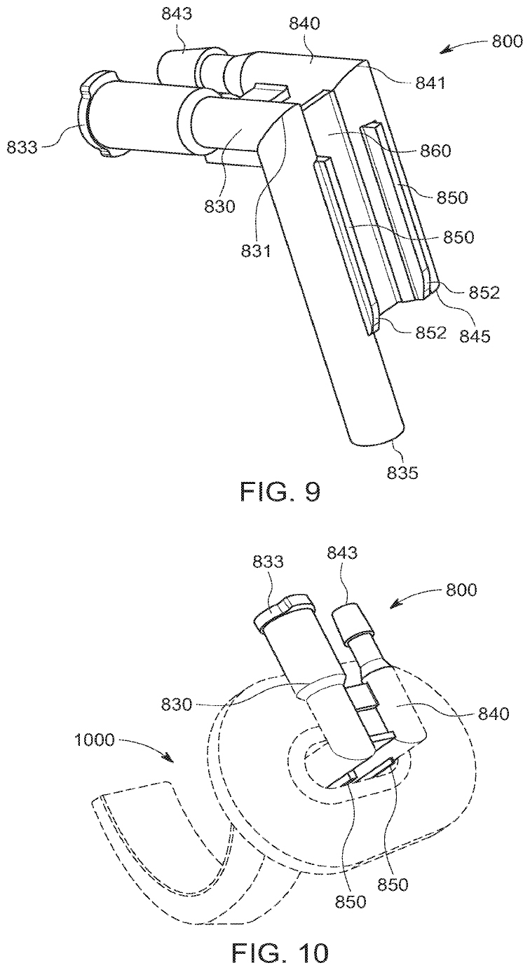

Referring to FIGS. 8-9, one embodiment of an airway adjunct 800 comprises gas sampling tube 830 and gas administration tube 840. According to embodiments, gas sampling tube 830 comprises a port 833 and an internal terminal end 835. According to embodiments, gas administration tube 840 comprises a port 843 and an internal terminal end 845.

In embodiments, internal terminal ends 835, 845, each comprise a different longitudinal position as set forth above with respect to internal terminal ends 135, 145. As depicted in FIGS. 8-9, gas sampling tube 830 and gas administration tube 840 are connected by web 860. According to various embodiments, web 860 has sufficient rigidity to hold gas sampling tube 830 and gas administration tube 840 in a spaced relationship relative to each other. Embodiments of airway adjunct 800 comprise ridges 850 on gas sampling tube 830 and gas administration tube 840, wherein ridges 850 have a sufficient size to allow airway adjunct 800 to snugly fit within the throat of an airway device. In some embodiments, ridges 850 comprise tapered ends 852, which may ease insertion of airway adjunct 800 into an airway device. Some embodiments comprise one or more ridges on web 860.

Various embodiments of airway adjunct 800 have elbow bends 831, 841 in gas sampling tube 830 and gas administration tube 840. In some embodiments, elbow bends 831, 841 comprise an angle of approximately ninety degrees. In other embodiments, elbow bends 831, 841 comprise angles of various other degrees as may be desirable.

As shown in FIG. 10, airway adjunct 800 may be seated within an oropharyngeal airway device 1000 to form an airway assembly. In embodiments, airway adjunct 800 may be held within the throat of oropharyngeal airway device 1000 by a friction fit between ridges 850 and a corresponding internal surface of oropharyngeal airway device 1000. In other embodiments, airway adjunct 800 lacks ridges but may be held in place within oropharyngeal airway device 1000 by a friction fit between various external-facing surfaces of airway adjunct 800 and corresponding internal-facing surfaces of oropharyngeal airway device 1000.

According to embodiments of the present disclosure, an airway adjunct may be adapted to fit a variety of oropharyngeal airway device types. As would be understood by a person of ordinary skill in the art having the benefit of this disclosure, airway adjuncts may comprise clamping tabs and/or friction fit surfaces as set forth above, adapted to fit throat diameters and/or internal shapes of different airway devices. Further, one airway adjunct embodiment may comprise clamping tabs and/or friction fit surfaces adapted to fit multiple different airway device collar sizes and/or shapes. As such, a single airway adjunct design or size may be stocked in a hospital or other operating facility, the single design or size being capable of universally fitting multiple different types or sizes of airway devices.

The present disclosure is not limited to clamping tabs for attaching adjunct devices to an airway device as described above. In other embodiments, the adjunct device is attached and/or fastened to an airway device in various other fashions. For example, some embodiments of an adjunct device are secured to an airway device by the use of magnets. Other embodiments secure to an airway device by hook-and-loop fasteners. Other embodiments are secure to an airway device by a tongue-and-groove configuration. In embodiments, the airway adjunct device is made of polyvinyl material. In other embodiments, the airway is manufactured from other materials and compounds thereof that may be appropriate for medical airway devices.

As depicted above, embodiments of airway adjunct devices have tubes with an external elbow bend. In other embodiments, one or more tubes have no such bends, but instead comprise a relatively straight section extending from the airway adjunct device body to a port. In other embodiments, the ports have other shapes and/or orientations as may be called for in various situations and circumstances. Some embodiments may include, in addition to a gas administration tube and a gas sampling tube, other tubes and/or ports providing other access as may be useful.

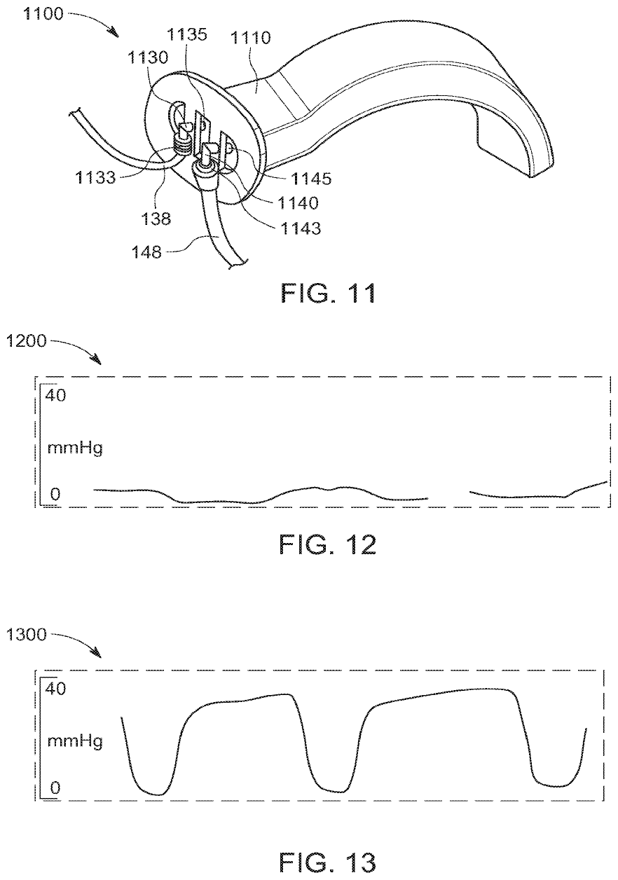

Referring to FIG. 11, embodiments of the present disclosure comprise an integral airway assembly 1100 having a gas sampling tube 1130 and a gas administration tube 1140. According to some embodiments, airway assembly 1100 comprises a single-piece component including both an airway adjunct portion and an airway device portion. In some examples, a single-piece airway assembly 1100 is manufactured as an integrally formed component. In other embodiments, an airway assembly 1100 comprises multiple separate components that may be permanently or separably joined. In one embodiment, gas sampling tube 1130 comprises port 1133. In one embodiment, gas administration tube 1140 comprises port 1143 In various embodiments, gas sampling tube 1130 and gas administration tube 1140 each comprise a respective hollow tubular element providing fluid communication between ports 1133, 1143 and corresponding internal terminal ends 1135, 1145. In embodiments, internal terminal ends 1135, 1145 terminate within housing 1110 of airway assembly 1100.

According to various embodiments, internal terminal ends 1135, 1145 are longitudinally offset from each other. As set forth above with respect to internal terminal ends 135, 145, in embodiments of the present disclosure, internal terminal end 1135 extends farther into the central cavity of housing 1110 than internal terminal end 1145. In some embodiments, the longitudinal offset between internal terminal end 1135 and internal terminal end 1145 is defined as an absolute difference as set forth above with respect to internal terminal ends 135, 145. In some embodiments, the longitudinal offset between internal terminal end 1135 and internal terminal end 1145 is defined as a relative difference as set forth above with respect to interior portions of tubes 130, 140. In embodiments shown and described in the present disclosure, and other embodiments that fall within the scope thereof, internal terminal ends 1135, 1145 may be longitudinally offset from each other.

Throughout the present disclosure, the term “longitudinal” may be defined as a direction parallel to the patient's airway flow. In other words, an airway flow direction is parallel to an airway device that is inserted, may be inserted, or has been inserted into a patient's airway. Particularly, the airway flow direction may be defined by the direction of bulk movement of air through a patient's internal respiratory pathway at or around the pharynx. Thus, the airway flow direction can be applied to describe the ports and tubing of an airway adjunct, even while the airway adjunct is not inserted into an airway device, based on the intended and designed orientation of insertion into an airway device.

Some embodiments of the present disclosure comprise airway adjunct devices that are adapted to fit a nasopharyngeal airway. Such embodiments are adapted to allow a healthcare provider to fasten the airway adjunct to the airway and thereby administer oxygen, analyze carbon dioxide and/or additional analysis, or complete other access functions as may be appropriate.

In operation, a healthcare provider may utilize an embodiment of an airway device or an airway adjunct during a medical procedure where it may be desirable to monitor a patient's carbon dioxide or other exhaled gases. Additionally, or alternatively, a healthcare provider may utilize an embodiment of an airway device or an airway adjunct during a medical procedure where it may be desirable to administer oxygen or other administered gas to the patient.

According to various embodiments, a healthcare provider can insert an airway device, such as an oropharyngeal airway device or a nasopharyngeal airway device, into a patient's airway following standard practices and procedures. Thereafter, the healthcare provider may secure an airway adjunct to the airway device. In alternative embodiments, the adjunct device may be secured to the airway device prior to insertion into the patient's airway. As set forth above, the airway adjunct may be secured to the airway device by a friction fit, by clamping tabs, magnets, hook-and-loop, tongue-and-groove, or other mechanisms as known in the art or not yet known in the art. In some embodiments, the healthcare provider may insert an integral airway assembly into the patient's airway, the airway assembly having a gas sampling tube and a gas administration tube.

Under some circumstances, it may be desirable for ports of the airway adjunct to point downward (i.e, toward the patient's chin). For example, a medical procedure may involve treating a portion of the patient's face above the mouth and for reasons of efficiency or convenience of the healthcare provider, and to mitigate interference and/or obstruction from tubes and ports, it may be desirable for tubing connected to the airway to be directed away from the target area on the patient. In such cases, the healthcare provider may attach the airway adjunct to the airway device with the appropriate orientation. In other cases, it may be desirable for ports of the airway to adjunct to point upward (i.e, toward the patient's nose). The healthcare provider may likewise attach the airway adjunct to the airway device with the appropriate orientation. In some cases, it may be desirable to switch the orientation of the ports partway through a medical procedure, which the healthcare provider may do. In some embodiments, the airway adjunct may be removed from the airway device by pushing or squeezing clamping tabs (or otherwise released the airway adjunct from the airway device) and pulling the airway adjunct away from the airway device. In other embodiments that do not include clamping tabs, the airway adjunct may be removed from the airway device by pulling the airway adjunct away from the airway device.

Upon insertion of the airway device or airway assembly and/or attachment of the airway adjunct, the healthcare provider may connect one or more ports of the airway adjunct or airway assembly to corresponding tubes. For example, a gas sampling tube of the airway adjunct or airway assembly may be connected to a gas sampling tube connected to expiration monitoring equipment such as a capnography equipment. A gas administration tube may be connected to a supply such as an oxygen supply line or oxygen tank.

Under some circumstances, it may be desirable to use an airway adjunct during a portion of a medical procedure. In such cases, the adjunct device can be inserted in place on the airway device or removed at any time during the procedure.

According to various embodiments of the present disclosure, the relative positions of internal terminal ends may affect the accuracy of exhaled gas monitoring and data gathering. For example, embodiments of the present disclosure comprise an airway assembly that has a gas sampling tube that extends farther into the body of the airway assembly than a gas administration tube. Such embodiments may undergo reduced diffusion of the patient's exhalation with administered gas compared to an airway having both ports at the same longitudinal position.

Referring to FIG. 12, a carbon dioxide waveform 1200 for a patient's exhalation is depicted. Waveform 1200 represents carbon dioxide monitored using an airway having respective internal terminal ends of an oxygen administration tube and a gas sampling tube port that are approximately even with each other within the airway assembly.

Referring to FIG. 13, a carbon dioxide waveform 1300 for a patient's exhalation is depicted. Waveform 1300 represents carbon dioxide monitored using an airway having an internal terminal end of a gas sampling tube that extends at least one millimeter farther into the airway assembly than an internal terminal end of a gas administration port.

As can be seen by comparing waveform 1200 with waveform 1300, carbon dioxide waveform 1200 is relatively dampened. It is theorized that diffusion with administered oxygen gas thus affected waveform 1200 and reduced the data resolution thereof.

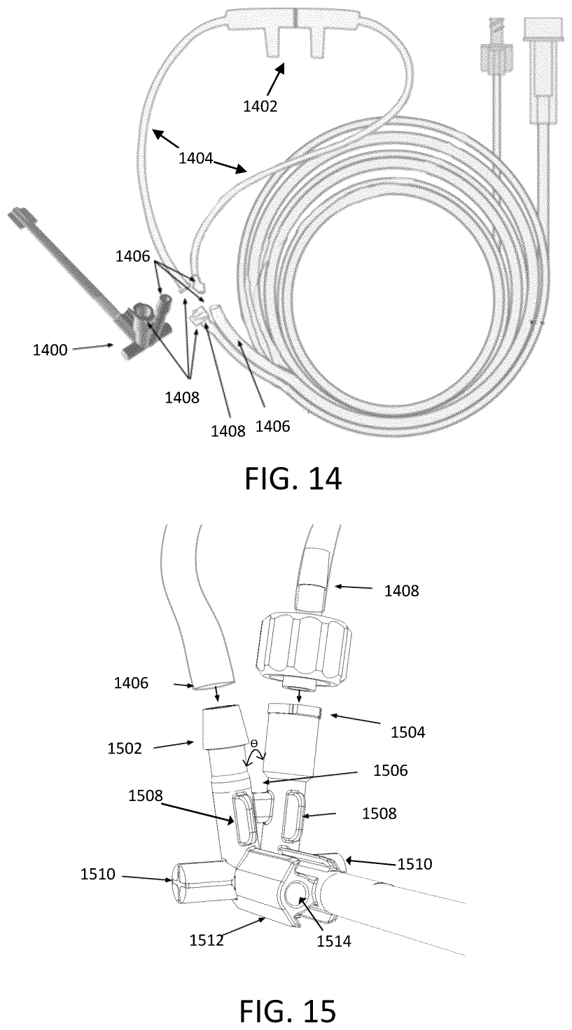

FIG. 14 depicts a side view of an adjunctive airway monitoring device (“AAMD”) 1400. Standard nasal prongs 1402 are connected to tubing 1404 with the tubing 1404 connecting to an oxygen supply line 1406 and a monitored gas line 1408. The tubing 1404 is disconnected from the oxygen supply line 1406 and monitored gas line 1408 and the oxygen supply line 1406 and the monitored gas line 1408 are connected to the AAMD 1400. In one embodiment, the oxygen supply line 1406 is connected to the tubing 1404 via a barbed connector on the end of the tubing 1404. Consistent with this embodiment, the oxygen supply line 1406 connects to a barded connector 1406 on the AAMD device 1400. In another embodiment, the oxygen supply line 1406 connects to the AAMD device 1400 using Leur connector. In one embodiment, the monitored gas line 1408 connects to the tubing 1404 using a Leur connector on the end of the tubing 1404. Consistent with this embodiment, the monitored gas line 1408 connects to the AAMD device 1400 with a Leur connector. In another embodiment, the monitored gas line 1408 connects to the AAMD device 1400 using a barbed connector.

FIG. 15 depicts a perspective view of the AAMD device 1400. The AAMD device 1400 includes a barbed connection unit 1502 and a Luer connection unit 1504. The barbed connection unit 1502 and Luer connection unit 1504 are separated by a separation unit 1505 that positions the barbed connection unit 1502 and Luer connection unit 1504 an angle theta (0) relative to one another. An offset unit 1508 is positioned on an outside surface of the barbed connection unit 1502 and the Luer connection unit 1504. The offset unit 1508 prevents the oxygen supply line 1406 and monitored gas line 1408 from extending beyond a desired point barbed connection unit 1502 and Luer connection unit 1504.

Gripping units 1510 are positioned below the barbed connection unit 1502 and the Luer connection unit 1504. The gripping units 1510 allow a user of the AAMD device 1400 to securely hold the AAMD device 1400 when tubes 1406 and 1408 are secured or removed from the AAMD device 1400. A vent unit 1512 is in fluid communication with the oxygen supply line 1406 via either the barbed connection unit 1502 or Luer connection unit 1504, based on which unit is connected to the oxygen supply line 1406. In one embodiment, the oxygen supply line 1406 is connected to the barbed connection unit 1502 and the vent unit 1512 is positioned at the bottom of the barbed connection unit 1502. The vent unit 1512 includes an opening 1514 that allows oxygen to exit the vent unit 1512 and into an external device via a tube connected to the vent 1512 and an input port on the external device.

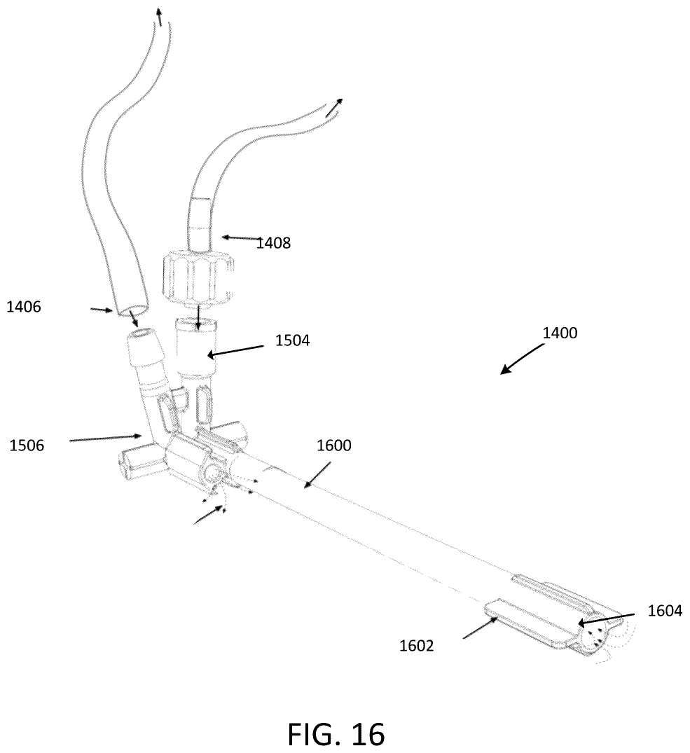

FIG. 16 depicts a perspective view of the AAMD device 1400. The AAMD device 1400 includes a flexible tube 1600 in fluid communication with the Luer connection unit 1504. The flexible tube 1600 can be a variety of lengths or diameters to fit all types of oral pharyngeal airway (OPA) or nasopharyngeal airway (NPA) units. The end of the flexible tube 1600 includes centering units 1602 adhered to the sides of the flexible tube 1602 to center the flexible tube 1602 in an OPA or NPA. The centering units 1602 may be formed as molded features, a secondary larger tube, molded standoffs, or a trumpet.

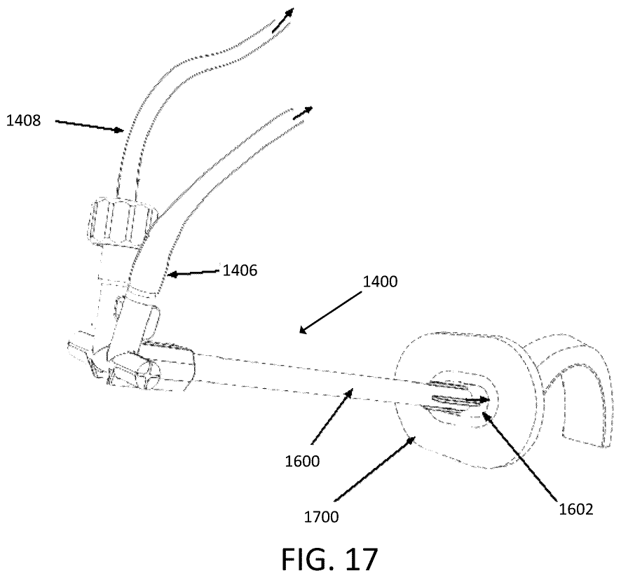

FIG. 17 depicts a perspective view of the AAMD device 1400 connected to an OPA device 1700. The OPA device 1700 is connected to the AAMD device 1400 with the centering units 1602 engaging openings in the back of the OPA device 1700. The OPA device 1700 is inserted into the mouth of a patient and ECO2 flows through the OPA device 1700 through the flexible tube 1600 of the AAMD device 1400 and into the monitored gas line 1408.

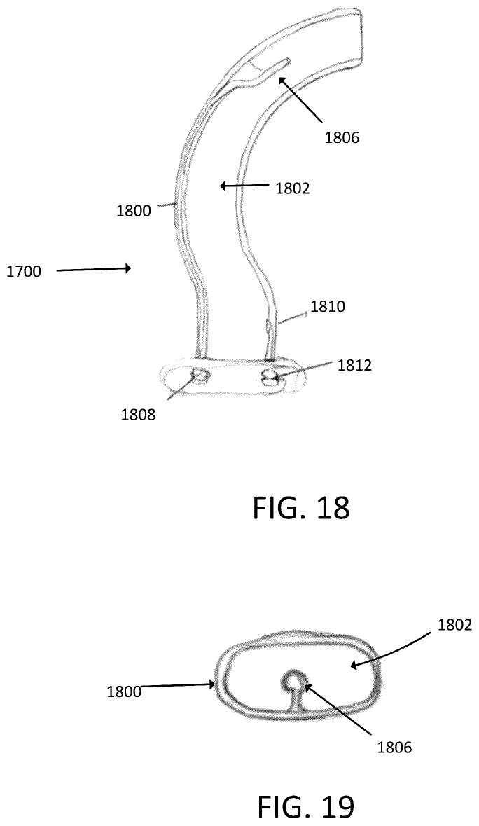

FIG. 18 depicts a perspective view of an OPA device 1700. The OPA device includes a monitored gas line 1800 that projects into a channel 1802 of the OPA device 1700. A distal end 1806 of the monitored gas line 1800 is positioned in channel 1802 such that ECO2 exhaled by the patient is directed into the distal end 1806 and through the tube 1800 to the opening 1808 that is connected to the AAMD device 1400. The OPA device 1600 includes an O2 exit opening 1810 and an O2 live port 1812. The AAMD device 1400 can connect to an OPA, as shown, or to NPAs both intraorally and intranasally. The AAMD device 1400 may be used as a standalone unit with the flexible tube 1602 being bent and positioned into the patient's mount much like a dental suction unit. The AAMD device may also be used on a base block for gastrointestinal devices. The AAMD device may also be incorporated into an OPA or NPA to create a standalone device. Because of the use of the flexible tube 1602, the distance between the OPA device 1700 and the monitored gas line 108 is increased and suctioned secretions are reduced. FIG. 19 depicts a cutaway view of the OPA device 1700. The distal end 1806 of the monitored gas line 1800 is positioned in the center of the OPA device 1400 channel 1802. By positioning the distal end in the center of the channel, the monitored gas can be collected and sent for analysis.

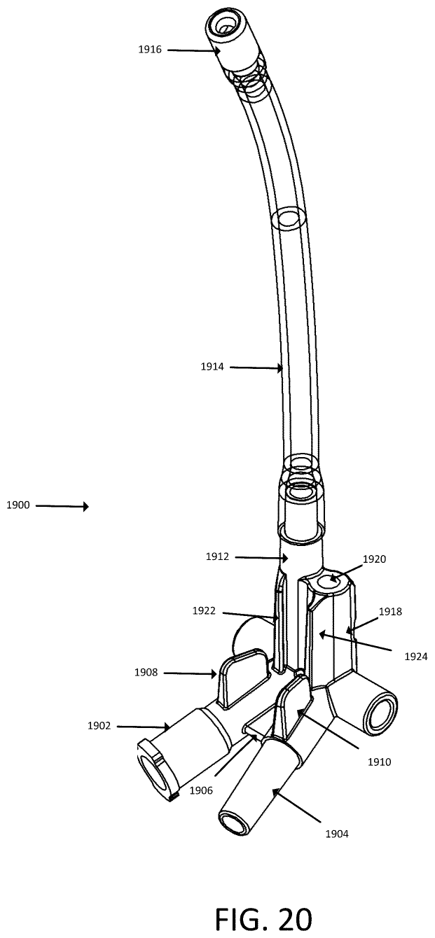

FIG. 20 depicts another embodiment of an AAMD 1900. The AAMD 1900 includes a gas monitoring port 1902 and an oxygen supply port 1904 separated from each other by a spacer 1906. Tabs 1908 and 1910 are positioned on the outer surface of the respective gas monitoring port 1902 and oxygen supply port 1904. The tabs 1908 and 1910 are configured to assist in removal of the AAMD 1900 from an OPA or NPA. The gas monitoring port 1902 includes an upper portion 1912 that is connected to a gas sampling line 1914. The gas sampling line 1914 is made from a substantially flexible material that allows the gas sampling line to follow the shape of the OPA or NPA into which the gas sampling line 1914 is placed. In one embodiment, a tip 1916 is affixed to the end of the gas sampling line 1914. In another embodiment, the tip 1916 of the gas sampling line 1914 is angled to prevent vapor lock from occurring. In one embodiment, the gas sampling line 1914 has a diameter of 4 mm. In another embodiment, the gas sampling line 1914 has a diameter of between 2-4 mm. In another embodiment, the gas sampling line 1914 has a diameter of between 3-4 mm. In another embodiment, the diameter of the gas sampling line is between 4-4.5 mm. In one embodiment, the gas sampling line 1914 has a length that is shorter than the length of the OPA or NPA into which the gas sampling line 1914 is inserted. In another embodiment, the gas sampling line 1914 is sized such that the gas sampling line 1914 is positioned in the center of the channel in the OPA or NPA. In one embodiment, the gas sampling line 1914 has a length of 56 mm. In another embodiment, the gas sampling line 1914 has a length of less than 56 mm.

The oxygen supply port 1904 includes an upper portion 1918 that includes an opening 1920 through which oxygen is passed into the OPA or NPA. The upper portion 1912 of the gas monitoring port 1902 and the upper portion 1918 of the oxygen supply port 1904 includes tabs 1922 and 1924. The tabs 1922 and 1924 are sized to engage the opening in the OPA or NPA to securely position the AAMD 1900 in the OPA or NPA. In one embodiment, the tabs 1922 and 1924 are angled towards each other. Notches 2002 are positioned on fins 2002 formed on the back of the upper portions 1912 and 1918 of the gas monitoring port 1902 and oxygen supply port 1904. The notches 2002 are configured to engage a bite block to secure the AAMD 1900 to the NPA and OPA securely.

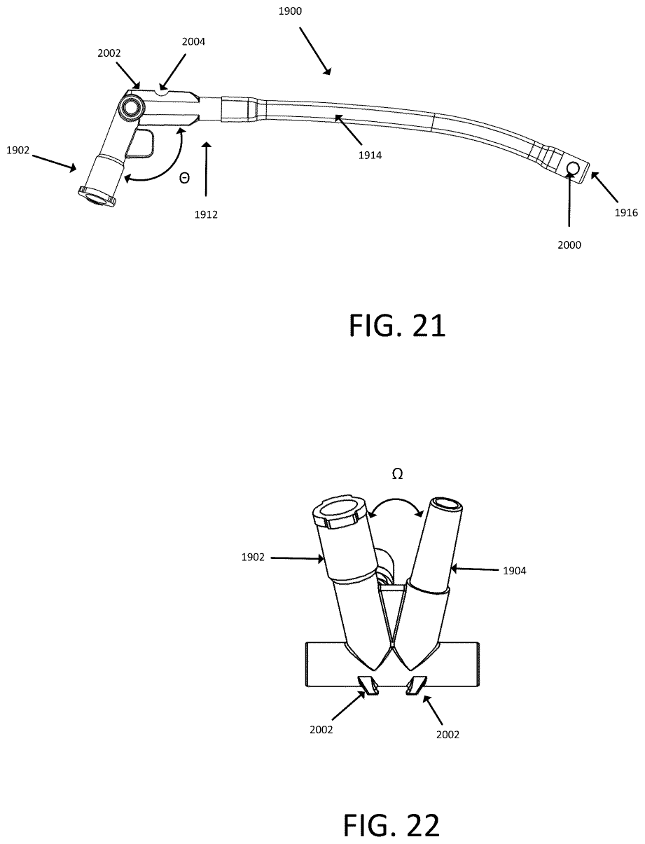

FIG. 21 depicts aside view of the AAMD 1900. The upper portion 1912 of the gas sampling port 1902 is angled from the lower portion of the gas sampling port 2902 by an angle Θ and the upper portion 1918 of the oxygen supply port 1904 is angled from the lower portion by the same angle Θ. The tip 1916 includes opening 2000 positioned on one side of the tip 1916. In one embodiment, the tip 1916 includes more than one opening 2000 on the side of the tip 1916. In another embodiment, the tip 1916 does not include any openings. FIG. 21 depicts a bottom view of the AAMD 1900. The lower portions of the gas sampling port 1902 and oxygen supply port 1904 are separated by spacer 1906 with spacer 1906 being triangle shaped that separates the gas sampling port 1902 from the oxygen supply port 1904 by an angle Ω.

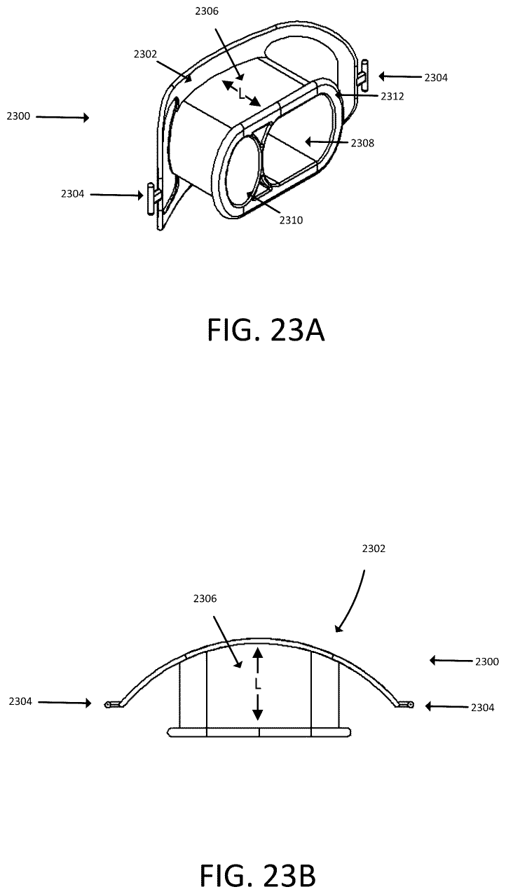

FIG. 23A depicts a perspective view of an endoscopy bite block 2300. The bite block 2300 includes a base portion 2302 with the base portion 2302 including two tabs 2304 on opposite sides of the base portion 2302. In one embodiment, the tabs 2304 are configured to connect to a strap that holds the bite block 2300 in place on a patient's body. A central portion 2306 extends from one side of base portion 2302 with the central portion including a first channel 2308 and a second channel 2310. The second channel 2310 is sized to accommodate an airway device that is inserted into a patient's throat during a surgical procedure. The central portion 2306 extends from the base portion 2302 a length L. Length L is sized to accommodate a patient's teeth such that the patient bites down on the central portion 2306 to secure the bite block 2300 in the patient's mouth. In one embodiment, the central portion 2306 is oval in shape. In another embodiment, the central portion 2306 is rectangular in shape. In one embodiment, the width of the central portion is 40.6 mm. In another embodiment, the width of the central portion 2306 is less than 40.6 mm. In another embodiment, the width of the bite block is greater than 40.6 mm. The end of the central portion 2306 includes ridge 2312 that prevents the teeth of a user from sliding over the ridge 2312 when the bite block 2300 is in use. FIG. 23B depicts a top view of bite block 2300. The base portion 2302 is curved and has an arc that follows the contours of a human face around the mouth such that the base portion 2302 is flush with the skin of a patient using the bite block 2300. In one embodiment, the base portion 2302 includes a plurality of extensions that engage the skin if the user when the bite block is in use to assist with the removal of the bite block 2300 from a user's mouth. The Length L represents the length of the longest distance from the base portion 2306 to the ridge 2312.

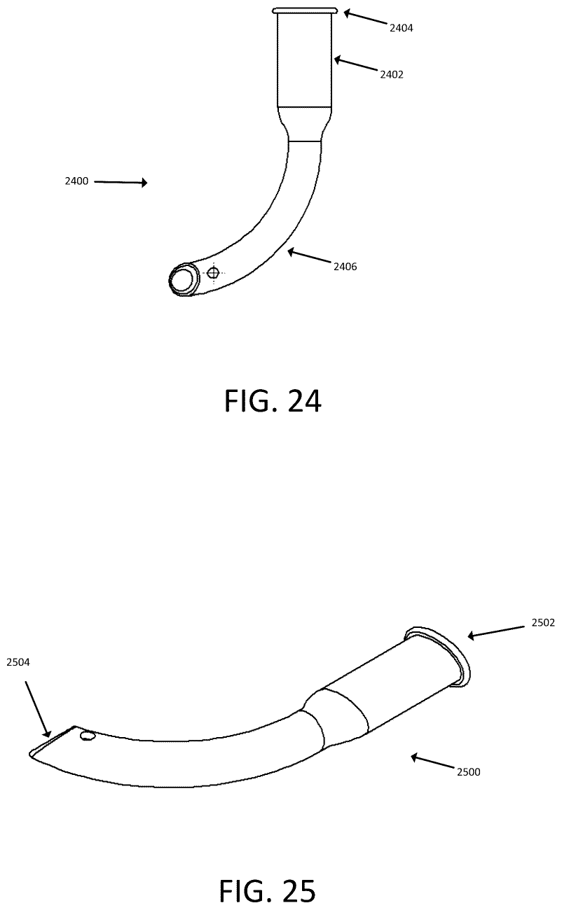

FIG. 24 depicts an airway device 2400. The airway device 2400 includes an upper portion 2402 that includes a ridge 2404 and a lower portion 2406. The lower portion 2406 is curved such that the curvature follows the curvature of an airway the airway device 2400 is inserted into during use. The airway device 2400 is made from a soft bendable material that allows the airway device 2400 to contour to different airway sizes without damaging the airway. The upper portion of the airway device 2402 is sized to engage an opening in an endoscopy bite block 2300 such that the airway device is held in place in the opening during use. A channel (nor shown) runs through the center of the airway device to allow air to flow to a patient during surgery. The opening at the top of the upper portion 2404 is sized to accommodate an airway device 1900. In one embodiment, the height from the ridge 2404 to the bottom edge of the tip 2408 of the airway device is 104.9 mm and the distance from the back of the ridge 2404 to the tip 2408 is 63.82 mm and the opening at the top of the upper portion 2402 is 24.49 mm.

FIG. 25 depicts a side perspective view of an airway device 2500. The opening 2502 at the top of the airway device 2500 is elliptical in shape with the tip 2504 of the airway device 2500 being aligned with the major axis of the elliptical opening. In another embodiment, tip 2504 of the airway device is aligned with the minor axis of the elliptical shape.

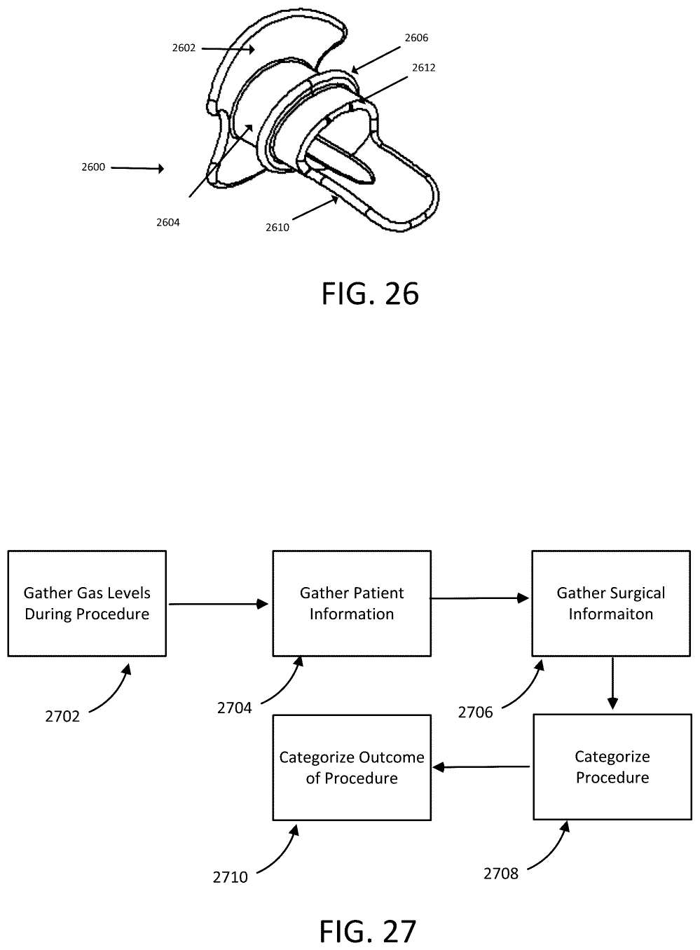

FIG. 26 depicts a perspective view of a simple bite block 2600. The bite block includes a base portion 2602 with a central portion 2604 extending from the base portion 2602 to a ridge 2606 on the central portion 2604. The ridge 2606 is positioned on the central portion 2604 such that the teeth of a user are held between the base portion 2602 and the ridge 2606. The central portion 2604 extends past the ridge with a lower portion 2610 of the base portion 2604 extending past an upper portion 2612 of the central portion 2604. The lower portion 2610 has a length sufficient to depress a user's tongue when the bite block is in the user's mouth. The bite block includes openings that are sized to accommodate an airway device 2500.

FIG. 27 depicts a process of gathering and storing information on a surgical procedure. In step 2702, gas levels are monitored via a monitoring unit connected to the AAMD 1900. Gas measurements are continuously measured throughout a surgical procedure and are stored in the memory of a computer. In step 2704 information on the patient undergoes in the procedure is gathered and stored in the memory of a computer. In one embodiment, the patient's information includes, but is not limited to, age, gender, ethnic background, medical history, medication being take, vital signs, physical condition and any other information on the patient. In step 2706, information on the surgical procedure is gathered. The information may include, but is not limited to, the type of procedure, complexity of the procedure, duration of the procedure, complications during the procedure, outcome of the procedure and any other information on the procedure. In step 2708, the procedure is categorized using the gas levels, patient information and surgical information. The categorization may include assessing the outcome and obstacles encountered during the procedure. In step 2710, the outcome of the procedure is categorized based on all of the information gathered on the procedure and patient.

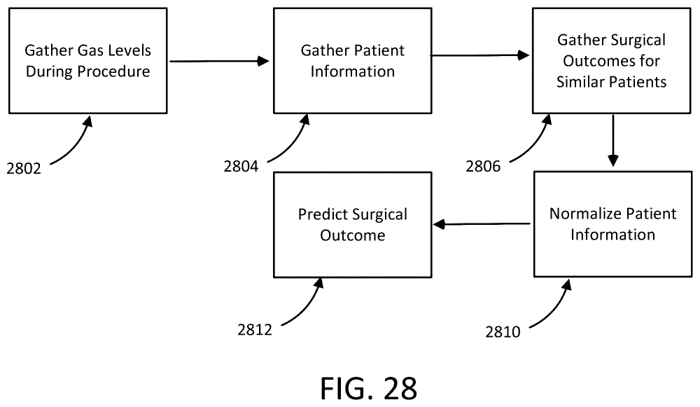

FIG. 28 depicts aa process performed to model a surgical procedure. In step 2802, the gas levels of a patient are monitored continuously during a procedure and are stored in memory. In step 2802, information on the patient is gathered and stored in memory. In step 2804, information from similar patients is retrieved from the memory. The system may gather information on a large number of patients having similar medical, physical and ethnic information as the patient undergoing the procedure. In step 2810, the system normalizes all the information on all prior patients including the continuous gas readings stored during each patient's surgical procedure. In normalizing the retrieved information, the system may apply weighting factors to each retrieved piece of information to extrapolate an adjusted value to compensate for differences between the current patient and the historical patient information.

In step 2812, the system makes a prediction on the surgical outcome based on the normalized data. In one embodiment, the system models the patient based on the normalized information to create a digital twin of the patient. The system monitors the gas levels via the AAMD and correlates gas levels and trends to the response by the digital twin of the patient to predict an outcome during the surgery. In one embodiment, the system notifies surgeons of the potential outcome prior to the outcome occurring and offers adjustments to the surgical procedure to compensate for the outcome.

As would be understood by a person of ordinary skill in the art having the befit of this disclosure, in office-based or ambulatory surgery, or other settings where minimally invasive anesthesia may be preferred, embodiments of the present disclosure may assist healthcare providers in administering oxygen, anesthesia, and/or additional substances to a patient while allowing for the collecting of exhalation for measuring and monitoring thereof.

Although the present disclosure is described in terms of certain preferred embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the spirit and scope of the present disclosure.

What is claimed:

1. An airway measurement device including:
a flexible tube having an opening at one end;
a monitored fluid unit connected to the flexible tube on an end opposite the opening;
an oxygen supply connection unit adjacent to the monitored fluid unit with the oxygen supply connection unit having a venting unit positioned parallel to the flexible tube; and
a gripping unit on the bottom the monitored fluid unit and the oxygen supply connection unit.

2. The airway measurement unit of claim 1 wherein the monitored fluid unit is offset from the oxygen supply unit by an angle.

3. The airway measurement unit of claim 1 including offset units positioned on the monitored fluid unit and the oxygen supply connection unit.

4. The airway measurement unit of claim 1 wherein an oxygen supply line is connected to the oxygen supply connection unit.

5. The airway measurement unit of claim 4 wherein oxygen from the oxygen supply unit flows through the oxygen supply connection unit and through the vent an external device via a tube.

6. The airway measurement unit of claim 1 including at least one centering unit on the flexible tube near the opening for connecting the flexible tube to an external device.

7. The airway measurement unit of claim 1 wherein the opening is connected to an oropharyngeal device.

8. The airway measurement unit of claim 1 including a monitored air line connected to the monitored air unit.

9. The airway measurement unit of claim 7 wherein the opening in the flexible tube connects to a monitoring air tube in the oropharyngeal device.

10. The airway measurement unit of claim 9 wherein the monitoring tube in the oropharyngeal device diverts carbon dioxide from the oropharyngeal device through the flexible tube to the monitored fluid unit.

11. A method of monitoring gases in an airway, the method including the steps of:
connecting the opening of a flexible tube to an external device;
passing a gas through the flexible tube to a monitored fluid unit connected to the flexible tube on an end opposite the opening;
venting oxygen via an oxygen supply connection unit adjacent to the monitored fluid unit with the oxygen supply connection unit having a venting unit positioned parallel to the flexible tube; and
gripping the monitored air unit via a gripping unit on the bottom the monitored fluid unit and the oxygen supply connection unit.

12. The method of claim 11 wherein the monitored fluid unit is offset from the oxygen supply unit by an angle.

13. The method of claim 11 including the step of positioning at least one offset unit on the monitored fluid unit and the oxygen supply connection unit.

14. The method of claim 11 including the step of connecting an oxygen supply line to the oxygen supply connection unit.

15. The method of claim 14 wherein oxygen from the oxygen supply unit flows through the oxygen supply connection unit and through the vent unit to via a tube.

16. The method of claim 11 including the step of positioning at least one centering unit on the flexible tube near the opening for connecting the flexible tube to an external device.

17. The method of claim 11 wherein the opening is connected to an oropharyngeal device.

18. The method of claim 11 including the step of connecting a monitored airline to the monitored air unit.

19. The method of claim 17 wherein the opening in the flexible tube connects to a monitoring air tube in the oropharyngeal device.

20. The method of claim 19 wherein the monitoring tube in the oropharyngeal device diverts carbon dioxide from the oropharyngeal device through the flexible tube to the monitored fluid unit.
